(19) United States
(12) Patent Application Publication
(10) Pub. No.: US 2014/0039275 A1
(43) Pub. Date: Feb. 6, 2014

(54) ELECTRONIC VITAL-SIGN SYSTEM WITH A MANUAL VITAL-SIGN DATA INPUT FUNCTION

(71) Applicant: OSTAR MEDITECH CORP., New Taipei City (TW)

(72) Inventor: Kuo-Yuan CHANG, New Taipei City (TW)

(73) Assignee: OSTAR MEDITECH CORP., New Taipei City (TW)

(21) Appl. No.: 13/958,259

(22) Filed: Aug. 2, 2013

(30) Foreign Application Priority Data
Aug. 3, 2012 (TW) 101214943

(51) Int. Cl. A61B 5/00

(52) U.S. Cl. CPC A61B 5/72
USPC 600/301

(57) ABSTRACT

The present invention discloses an electronic vital-sign system with a manual vital-sign data input function, wherein a vital-sign recording unit is added to an electronic vital-sign system and used to input vital-sign data manually. The electronic vital-sign system detects vital-sign data automatically and receives other vital-sign data manually input by the user, such as the data of at least one of respiration rate, blood glucose, body temperature, body weight, food intake volume, fluid infusion volume, urine volume, stool volume and pain score. The system transmits the detected vital-sign data and manually-input vital-sign data to a cloud database where the vita-sign data is processed and integrated. The present invention incorporates a manual vital-sign data input function with the original vital-sign detection function and far-end transmission function of an electronic vital-sign system to provide appropriate assistance for hospitals, telecare centers, elderly nursing centers, elderly communities, and senior citizens living alone.

### BACKGROUND OF THE INVENTION

1. Field of the Invention

The present invention relates to an electronic vital-sign system with a manual vital-sign data input function, particularly to a multi-functional electronic vital-sign system, such as a blood pressure monitoring system, a blood oxygen monitoring system, a blood glucose monitoring system, an electrocardiograph monitoring system or a patient monitor, which is linked to a cloud database.

2. Description of the Related Art

Medical personnel need to measure and record basic vital signs of inpatients, including pulse, heart rate, respiration rate, blood pressure, blood oxygen, body temperature and electrocardiograph. In the past, medical personnel record vital signs manually. The modern hospitals normally adopt EMR (Electronic Medical Records) and use electronic physiological monitoring systems to automatically transmit vital-sign data of patients. However, most of the vital signs still need to be measured, transcribed and then input into computers by medical personnel manually. Therefore, the present invention proposes an electronic vital-sign system with a manual vital-sign data input function, which not only saves medical personnel the time of transcription but also avoid inaccuracy of manual transcription and errors of vital-sign data transference. Because of population aging and low birthrate, there are more and more senior citizens living alone and elderly communities in our society. In addition to miniature electronic devices for measuring basic vital signs, such as pulse, heart rate and blood pressure, elderly communities also need more sophiscated far-end electronic vital-sign systems to cooperate with the healthcare systems of hospitals. In a cloud healthcare system for senior citizens, the caregivers or family members daily measure their pulse, heart rate, respiration rate, blood pressure, blood oxygen, blood glucose and/or electrocardiograph with the available medical devices and input the vital-sign data to the system, whereby the healthcare system can record and track their health statuses daily.

As the ordinary electronic vital-sign measurement devices are easy to carry and easy to operate, they are very popular in hospitals, clinics, elderly nursing centers, and nursing homes. However, medical personnel can neither manually input vital-sign data into the ordinary electronic vital-sign measurement devices nor link the ordinary electronic vital-sign measurement devices to cloud databases. Therefore, the ordinary electronic vital-sign measurement devices cannot automatically monitor the patients needing emergent nursing or the elders needing special nursing.

The medical personnel of hospitals will work more easily and efficiently if they can further manually input vital-sign data into ordinary electronic vital-sign measurement devices (such as electronic devices for measuring pulse, heart rate, respiration, blood pressure, blood oxygen, blood glucose, body temperature, body weight and electrocardiograph) in realtime and then transmit the vital-sign data to cloud databases while measuring vital signs of patients.

The telecare centers, which have adopted ordinary electronic vital-sign measurement devices to measure vital signs, such as pulse, heart rate, respiration, blood pressure, blood oxygen, blood glucose, body temperature and electrocardiograph, will be able to monitor and administrate the vital-sign data of patients, elders and testees in real time if the nursing personnel can further manually input vital-sign data into the ordinary electronic vital-sign measurement devices and then transmit the vital-sign data to cloud databases.

Accordingly, the Inventors develop an easy-to-operate electronic vital-sign system with a manual vital-sign data input function, which can monitor the pulse, heart rate and respiration of patients, elders and testees in realtime and transmit the measured vital-sign data and heart spectrum and the respiration rate manually input via a vital-sign recording unit to a cloud database, whereby the medical personnel can work more easily and efficiently.

### SUMMARY OF THE INVENTION

One objective of the present invention is to provide an electronic vital-sign system with a manual vital-sign data input function, which not only has a measurement function but also has a manual vital-sign data input function, and which further has a far-end cloud healthcare function, wherein the system links to a cloud database, a cloud HIS (Hospital Information System) or a cloud NIS (Nursing Information System), whereby the system can provide appropriate assistance for patients, elders and testees.

To achieve the abovementioned objective, one embodiment of the present invention proposes an electronic vital-sign system with a manual vital-sign data input function, which comprises a detection unit, a vital-sign recording unit, a calculating/processing unit, a display unit, a transmission unit, and a power unit. The detection unit detects first vital-sign information. The user uses the vital-sign recording unit to input second vital-sign information manually. The calculating/processing unit connects with the detection unit and the vital-sign recording unit and processes the first vital-sign information or the second vital-sign information. The display unit connects with the calculating/processing unit and displays the first vital-sign information or the second vital-sign information. The transmission unit connects with the calculating/processing unit and transmits the first vital-sign information or the second vital-sign information to a cloud database. The power unit connects with the detection unit, the vital-sign recording unit, the calculating/processing unit, the display unit and the transmission unit, and provides power for these units or charges the system.

In one embodiment, the first vital-sign information is information of at least one of pulse, heart rate, blood pressure, blood oxygen, blood glucose, body temperature, and body weight; the second vital-sign information is information of at least one of respiration rate, blood glucose, body temperature, body weight, food intake volume, fluid infusion volume, urine volume, stool volume and pain score.

In one embodiment, the calculating/processing unit can process data to obtain the heart information.

In one embodiment, the vital-sign recording unit is realized by a function button, which can be pressed or rotated to adjust the value so as to input one or more types of the second vital-sign information.

In one embodiment, the vital-sign recording unit is realized by more than one function button, which can be pressed or rotated to adjust the value so as to input one or more types of the second vital-sign information.

In one embodiment, the vital-sign recording unit is realized by a touchscreen. The user clicks on the option items of the touchscreen to adjust the value so as to input one or more types of the second vital-sign information.

In one embodiment, the transmission unit is a wired transmission interface, such as a USB port or an RS232 port.

In one embodiment, the transmission unit is a wireless transmission interface, such as a 2.4G or WiFi transmission interface.

In one embodiment, the electronic vital-sign system with a manual vital-sign data input function is connected with an ID scanner, such as a bar-code scanner, a card reader, an RFID (radio frequency identification) reader, or an NFC (Near Field Communication) device.

One embodiment of the present invention further proposes another electronic vital-sign system with a manual vital-sign data input function, which comprises a detection unit, a calculating/processing unit, a connection interface, a power unit and an external electronic device able to transmit information. The detection unit detects first vital-sign information. The calculating/processing unit connects with the detection unit and processes the first vital-sign information. The connection interface connects with the calculating/processing unit. The power unit connects with the detection unit, the calculating/processing unit and the connection interface, and provides power for these units or charges the system. The external electronic device connects with the calculating/processing unit through the connection interface. The user can use the external electronic device to manually input second vital-sign information. The external electronic device can transmit the first vital-sign information or the second vital-sign information to a cloud database. The electronic vital-sign system with a manual vital-sign data input function is connected with an ID scanner, such as a bar-code scanner, a card reader, an RFID (radio frequency identification) reader, or an NFC (Near Field Communication) device.

In one embodiment, the first vital-sign information is information of at least one of pulse, heart rate, blood pressure, blood oxygen, blood glucose, body temperature, and body weight; the second vital-sign information is information of at least one of respiration rate, blood glucose, body temperature, body weight, food intake volume, fluid infusion volume, urine volume, stool volume and pain score.

In one embodiment, the calculating/processing unit can process data to obtain the heart information.

In one embodiment, the external electronic device able to transmit information includes one or more function buttons, which the user uses to input one or more types of the second vital-sign information.

In one embodiment, the external electronic device able to transmit information includes a touchscreen, which the user uses to input one or more types of the second vital-sign information.

In one embodiment, the external electronic device able to transmit information is a notebook computer or a tablet computer.

In one embodiment, the external electronic device able to transmit information is a PDA (Personal Digital Assistant) or a smart phone.

In one embodiment, the heart information is information of an electrocardiograph or information of a heart spectrum. The heart spectrum is obtained via measuring the heart rate signals and transforming the heart rate signals into a frequency spectrum diagram with an FFT (Fast Fourier Transform) algorithm. The frequency spectrum diagram normally has 3-5 main frequency waveforms. The first main frequency waveform corresponds to heart rate frequency. If there are also several intricate frequency waveforms appearing in the main frequency spectrum, it indicates that the heart rate status appears irregular and can be considered abnormal. Therefore, the heart spectrum can be used to evaluate the function of the heart. The related technology has been disclosed in a U.S. Pat. No. 7,135,000 proposed by the Inventors and will not repeat herein.

Below, embodiments are described in detail in cooperation with the attached drawings to make easily understood the objectives, technical contents, characteristics and accomplishments of the present invention.

### BRIEF DESCRIPTION OF THE DRAWINGS

FIG. 1 is a block diagram schematically showing the architecture of an electronic vital-sign system with a manual vital-sign data input function according to one embodiment of the present invention; and

FIG. 2 is a block diagram schematically showing the architecture of the architecture of an electronic vital-sign system with a manual vital-sign data input function according to another embodiment of the present invention.

### DETAILED DESCRIPTION OF THE INVENTION

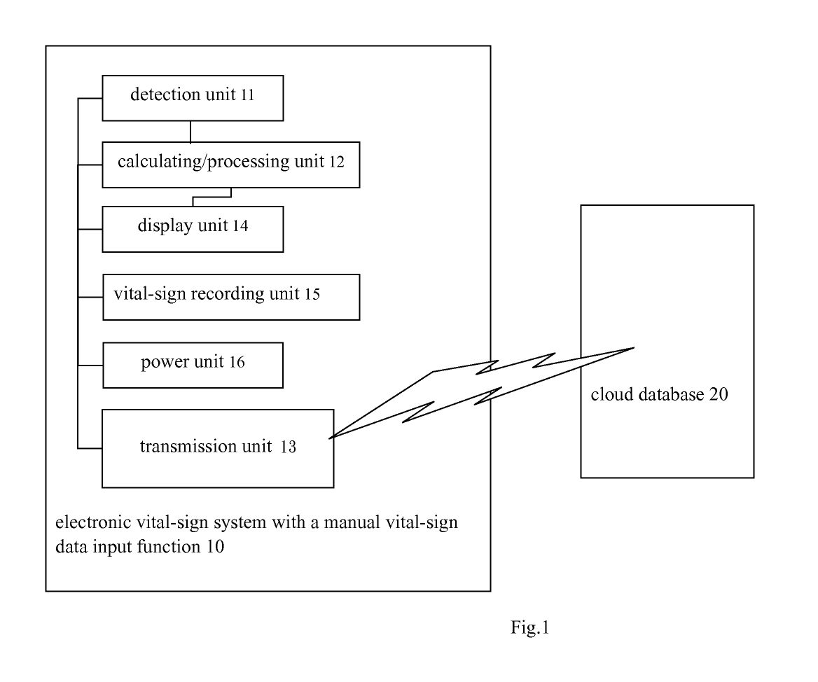

Refer to FIG. 1 a block diagram schematically showing the architecture of an electronic vital-sign system with a manual vital-sign data input function according to one embodiment of the present invention. As shown in FIG. 1, the electronic vital-sign system with a manual vital-sign data input function 10 of the present invention comprises a detection unit 11, a vital-sign recording unit 15, a calculating/processing unit 12, a display unit 14, a transmission unit 13, and a power unit 16. The detection unit 11 detects first vital-sign information. The user uses the vital-sign recording unit 15 to input second vital-sign information manually. The calculating/processing unit 12 connects with the detection unit 11 and the vital-sign recording unit 15 and processes the first vital-sign information or the second vital-sign information. The display unit 14 connects with the calculating/processing unit 12 and displays the first vital-sign information or the second vital-sign information. The transmission unit 13 connects with the calculating/processing unit 12 and transmits the first vital-sign information or the second vital-sign information to a cloud database 20. The power unit 16 connects with the detection unit 11, the calculating/processing unit 12, the display unit 14, the transmission unit 13 and the vital-sign recording unit 15, and provides power for these units or charges the system.

In one embodiment, the vital-sign recording unit 15 is realized by a function button, which can be pressed or rotated to adjust the value so as to input one or more types of the second vital-sign information. In one embodiment, the vital-sign recording unit 15 is realized by more than one function button, which can be pressed or rotated to adjust the value so as to input one or more types of the second vital-sign information. In one embodiment, the vital-sign recording unit 15 is realized by a touchscreen. The user clicks on the option items of the touchscreen to adjust the value so as to input one or more types of the second vital-sign information. In one embodiment, the transmission unit 13 is a wired transmission interface, such as a USB port or an RS232 port. In one embodiment, the transmission unit 13 is a wireless transmission interface, such as a 2.4G or WiFi transmission interface. In one embodiment, the electronic vital-sign system with a manual vital-sign data input function 10 is connected with an ID scanner, such as a bar-code scanner, a card reader, an RFID (radio frequency identification) reader, or an NFC (Near Field Communication) device.

In one embodiment, the electronic vital-sign system with a manual vital-sign data input function 10 is a pulse, heart rate and blood pressure measurement system. The detection unit 11 detects the pulse or heart rate information of a patient or a testee. The calculating/processing unit 12 processes the pulse or heart rate information to obtain an electrocardiograph, a heart spectrum or a blood pressure. The user may use the vital-sign recording unit 15 (such as a touchscreen or one or more function buttons) to input one or more pieces of information of respiration rates of patients or testees. The obtained information of pulse, heart rate, electrocardiography, blood pressure, or respiration rate is transmitted via the calculating/processing unit 12 and the transmission unit 13 to the cloud database 20 for fully automatic integration and analysis of the vital-sign data and personal data. The cloud database 20 is an ordinary cloud database, a cloud HIS (Hospital Information System) or a cloud NIS (Nursing Information System). After receiving the vital-sign information, the cloud database 20 undertakes data processing and establishes records, which are to be browsed by testees, family members, physicians or nursing personnel. The obtained information of electrocardiographs, heart spectra, blood pressures, and or respiration rates can be presented on the display unit 14, such as an LCD device or an LED display device. The power unit 16 is an alkaline battery assembly, a rechargeable battery assembly, a capacitor or a power supply device, providing power for these units or charging the system.

Besides, the electronic vital-sign system with a manual vital-sign data input function 10 may be a single-function vital-sign measurement system or a multi-function patient monitor.

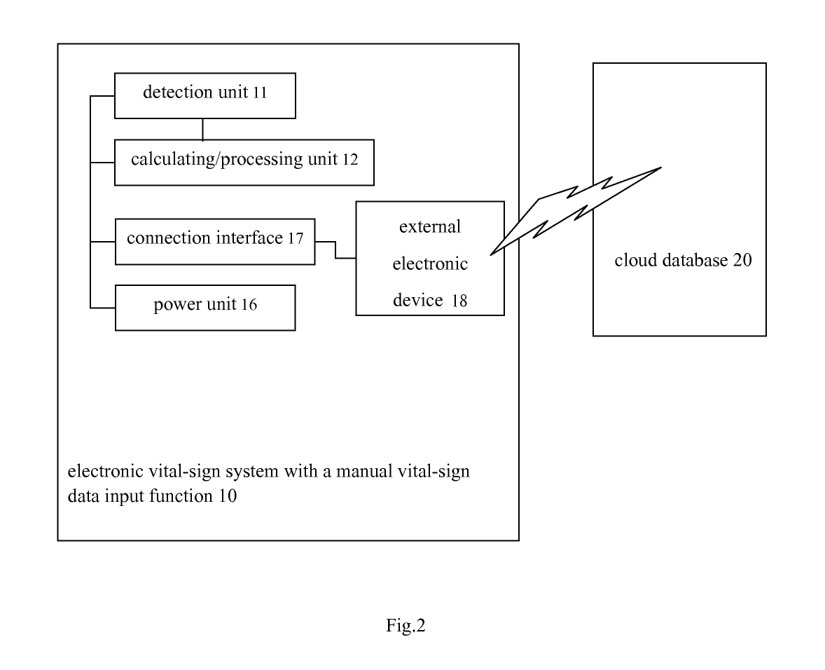

Refer to FIG. 2 a block diagram schematically showing the architecture of an electronic vital-sign system with a manual vital-sign data input function according to another embodiment of the present invention. As shown in FIG. 2, the electronic vital-sign system with a manual vital-sign data input function 10 of the present invention comprises a detection unit 11, a calculating/processing unit 12, a connection interface 17, a power unit 16, and an external electronic device 18 able to transmit information. The detection unit 11 detects first vital-sign information. The calculating/processing unit 12 connects with the detection unit 11 and processes the first vital-sign information. The connection interface 17 connects with the calculating/processing unit 12. The power unit 17 connects with the detection unit 11, the calculating/processing unit 12 and the connection interface 17, and provides power for these units or charges the system. The external electronic device 18 is able to transmit information and connected with the calculating/processing unit 12 via the connection interface 17. The user uses the external electronic device 18 to input second vital-sign information manually. Further, the user uses the external electronic device 18 to transmit the first vital-sign information or the second vital-sign information to a cloud database 20.

In one embodiment, the external electronic device 18 able to transmit information includes a function button, which the user uses to input one or more types of the second vital-sign information.

In one embodiment, the external electronic device 18 able to transmit information includes more than one function button, which the user uses to input one or more types of the second vital-sign information.

In one embodiment, the external electronic device 18 able to transmit information includes a touchscreen, which the user uses to input one or more types of the second vital-sign information.

In one embodiment, the external electronic device 18 able to transmit information is a notebook computer or a tablet computer.

In one embodiment, the external electronic device 18 able to transmit information is PDA (Personal Digital Assistant) or a smart phone.

In one embodiment, the electronic vital-sign system with a manual vital-sign data input function 10 is connected with an ID scanner, such as a bar-code scanner, a card reader, an RFID (radio frequency identification) reader, or an NFC (Near Field Communication) device.

In one embodiment, the electronic vital-sign system with a manual vital-sign data input function 10 is a pulse, heart rate and blood pressure measurement system. The detection unit 11 detects the pulse or heart rate information of a patient or a testee. The calculating/processing unit 12 processes the pulse or heart rate information to obtain an electrocardiograph, a heart spectrum or a blood pressure. Further, the user uses the external electronic device 18 able to transmit information to input the respiration rate of a patient. For example, the external electronic device 18 is realized by a touchscreen or one or more function buttons, which the user uses to input one or more pieces of information of respiration rates. The obtained information of pulse, heart rate, heart spectrum electrocardiograph, blood pressure, or respiration rate is transmitted via the calculating/processing unit 12, the connection interface 17 and the external electronic device 18 to the cloud database 20 for fully automatic integration and analysis of the vital-sign data and personal data. The cloud database 20 is an ordinary cloud database, a cloud HIS (Hospital Information System) or a cloud NIS (Nursing Information System). After receiving the vital-sign information, the cloud database 20 undertakes data processing and establishes records, which are to be browsed by testees, family members, physicians or nursing personnel. The obtained information of pulse, heart spectrum, electrocardiograph, blood pressure, or respiration rate can be presented on the external electronic device 18, such as an LCD device or an LED display device. The power unit 16 is an alkaline battery assembly, a rechargeable battery assembly, a capacitor or a power supply device, providing power for these units or charging the system.

Besides, the electronic vital-sign system with a manual vital-sign data input function 10 may be a single-function vital-sign measurement system or a multi-function patient monitor.

As described hereinbefore, the present invention adds a function of manually inputting second vital-sign information and a function of transmitting information to an ordinary electronic vital-sign system. Thereby, the present invention can instantly provide the vital-sign measurement data of patients, elders or other testees who need routine healthcare or immediate medical assistance. Further, the cloud database 20 enables the testees, family members or nursing personnel to track vital-sign information in a wired or wireless way (such as via the Internet), whereby is realized a far-end healthcare system.

The present invention proposes an electronic vital-sign system with a manual vital-sign data input function 10, which can fast and precisely detect and record vital-sign data of patients, elders or testees who need routine healthcare or immediate medical assistance and can instantly provide the vital-sign data for family members, telecare centers, personnel of nursing stations or physicians. Suppose that an abnormal state appears in a patient whose vital signs are being continually monitored by the electronic vital-sign system with a manual vital-sign data input function 10, such as a blood pressure monitoring system, a blood oxygen monitoring system, a blood glucose monitoring system, a patient monitoring system or an electrocardiograph monitoring system. In such a case, the related personnel should enquire the patient about his feeling as soon as possible and instantly input the abnormal vital-sign values into the system manually. The related personnel should send the patient to a large-scale hospital or medical center for further examination if necessary.

In conclusion, the present invention proposes an electronic vital-sign system with a manual vital-sign data input function, wherein a vital-sign detection function, an information transmission function, a manual vital-sign data input function and a far-end healthcare function are integrated in a system to provide appropriate assistance for patients, elders and testees.

What is claimed is:

1. An electronic vital-sign system with a manual vital-sign data input function, comprising
a detection unit detecting first vital-sign information;
a vital-sign recording unit used to input second vital-sign information;
a calculating/processing unit connecting with said detection unit and said vital-sign recording unit, and processing said first vital-sign information or said second vital-sign information;
a display unit connecting with said calculating/processing unit and displaying said first vital-sign information or said second vital-sign information;
a transmission unit connecting with said calculating/processing unit and transmitting said first vital-sign information or said second vital-sign information to a cloud database; and
a power unit connecting with said detection unit, said calculating/processing unit, said display unit, said transmission unit and said vital-sign recording unit, and providing power for said units or charging said system.

2. The electronic vital-sign system with a manual vital-sign data input function according to claim 1, wherein said first vital-sign information is information of at least one of pulse, heart rate, blood pressure, blood oxygen, blood glucose, body temperature, and body weight.

3. The electronic vital-sign system with a manual vital-sign data input function according to claim 1, wherein said second vital-sign information is information of at least one of respiration rate, blood glucose, body temperature, body weight, food intake volume, fluid infusion volume, urine volume, stool volume and pain score.

4. The electronic vital-sign system with a manual vital-sign data input function according to claim 1, wherein said vital-sign recording unit is realized by one or more function buttons used to input one or more types of said second vital-sign information.

5. The electronic vital-sign system with a manual vital-sign data input function according to claim 1, wherein said vital-sign recording unit is realized by a touchscreen used to input one or more types of said second vital-sign information.

6. An electronic vital-sign system with a manual vital-sign data input function, comprising
a detection unit detecting first vital-sign information;
a calculating/processing unit connecting with said detection unit and processing said first vital-sign information;
a connection interface connecting with said calculating/processing unit;
a power unit connecting with said detection unit, said calculating/processing unit and said connection interface, and providing power for said interface and said units or charging said system; and
an external electronic device able to transmit information, connected with said calculating/processing unit via said connection interface, used to manually input second vital-sign information, and transmitting said first vital-sign information or said second vital-sign information to a cloud database.

7. The electronic vital-sign system with a manual vital-sign data input function according to claim 6, wherein said first vital-sign information is information of at least one of pulse, heart rate, blood pressure, blood oxygen, blood glucose, body temperature, and body weight.

8. The electronic vital-sign system with a manual vital-sign data input function according to claim 6, wherein said second vital-sign information is information of at least one of respiration rate, blood glucose, body temperature, body weight, food intake volume, fluid infusion volume, urine volume, stool volume and pain score.

9. The electronic vital-sign system with a manual vital-sign data input function according to claim 6, wherein said external electronic device able to transmit information includes one or more function buttons used to input one or more types of said second vital-sign information.

10. The electronic vital-sign system with a manual vital-sign data input function according to claim 6, wherein said external electronic device able to transmit information includes a touchscreen used to input one or more types of said second vital-sign information.

11. The electronic vital-sign system with a manual vital-sign data input function according to claim 6, wherein said external electronic device able to transmit information is a notebook computer or a tablet computer.

12. The electronic vital-sign system with a manual vital-sign data input function according to claim 6, wherein said external electronic device able to transmit information is PDA (Personal Digital Assistant) or a smart phone.
